(19) United States
(12) Patent Application Publication
(10) Pub. No.: US 2010/0298700 A1
(43) Pub. Date: Nov. 25, 2010

(54) DEVICE FOR DETECTING THE DISINTEGRATION OF RADIOISOTOPES IN BIOLOGICAL TISSUE

(75) Inventors: Laurent Pinot, Lardy (FR); Jeremy Godart, La Celle Saint-Cloud (FR); Pierre-Auguste-Robert Delpierre, La Ciotat (FR); Philippe-Pierre-Louis Laniece, Saint-Aubin (FR); Bernard Dinkespiler, La Bedoule (FR)

Correspondence Address: BIRCH STEWART KOLASCH & BIRCH, PO BOX 747, FALLS CHURCH, VA, 22040-0747, US

(73) Assignee: Centre National De La Recherche Scientifique, Paris Cedex 16 (FR)

(21) Appl. No.: 12/682,557

(22) PCT Filed: Oct. 8, 2008
(86) PCT No.: PCT/FR2008/001407

§ 371 Date: Jul. 1, 2010

(30) Foreign Application Priority Data
Oct. 12, 2007 (FR) 07/07176

(51) Int. Cl. A61B 6/00

(52) U.S. Cl. 600/436

(57) ABSTRACT

Device that can be implanted into the brain, in particular that of a small animal (16), for the detection of radiation emitted by disintegration of a radioisotope, characterized in that it comprises an implantable detector (10) made of a semiconductor material and comprising a number of individual detectors (22), the device also including a system (14) of amplification, shaping, counting and wireless remote transmission circuits that are connected to the individual detectors (22) and are intended to be worn by the animal (16), the latter being awake and free to move.

This invention relates to a device for detecting the disintegration of radioisotopes present in biological tissues or organs, of an experimental animal, in particular.

The increasingly significant development of animal models mimicking human diseases such as neurodegenerative or tumorous diseases has opened up new opportunities in research conducted in fields as varied as toxicology, pharmacology, or even molecular biology. However, in vivo monitoring of these diseases requires the development of imaging techniques adapted to the specific constraints of studies on small animals, e.g., such as rodents.

For this purpose, the majority of anatomical and functional imaging modalities currently used clinically for humans have been adapted to small animals. Among these techniques, Positron Emission Tomography (hereinafter referred to as PET) attracts a strong interest since it is the only one to offer nearly picomolar sensitivity, thereby enabling studies to be carried out with regard to biochemical or molecular processes, without altering the normal or pathological conditions of the tissues or organs studied.

This imaging technique includes the intravenous injection of a radioactive tracer. This biologically active tracer attaches itself to the tissues of interest. The disintegration of the radioactive atom present in the tracer results in the emission of β+ radiation, which, after annihilation with an electron of the medium, produces two opposing γ rays, which are detected in coincidence by sensors situated outside of the animal.

At the present time, known devices exhibit limits specific to the technology used. As a matter of fact, they require the animal to be anaesthetized, which must be immobilised in order to be positioned between the sensors. The use of an anaesthetic adversely affects the physiological parameters of the animal's body and therefore complicates analysis of the biological activity of the tracer. In addition, with a view to the use in humans of molecules tested in animals, it is preferable to approximate clinical conditions, in which generally no anaesthia is administered. These tomographs likewise have low-sensitivity, due to the necessity of detecting two rays in coincidence and to the low solid angles resulting from the ring geometry of the sensors. This low sensitivity gives them low temporal resolution, which is poorly suited to the kinetic parameters from which numerous physiological parameters arise. Finally, these sensors are extremely expensive.

Two new approaches are currently being developed to remedy the aforementioned disadvantages.

The first one consists of a PET mini-camera specifically developed for imaging of the rat brain and intended to be fastened onto the head of the animal. This device is based on the use of avalanche photodiodes, specific electronics being associated with each sensing element. It surrounds the animal's head and includes a counter-weight system which compensates for the weight of the sensor. This device is relatively bulky and heavy, due to the weight of the sensor being approximately 150 g and to the necessity of having a physical link between the sensor and an analysis and storage unit, which does not enable complete freedom to be provided to the animal during the experiment and which limits the behavioural studies that can be carried out. Finally, since the travel of the β+ particles into the tissues is limited, the information comes from the coincidence detection of the two γ rays resulting from the annihilation of a β+ particle with the anti-particle thereof, the electron, by means of a ring of detectors situated to the exterior of the animal, which gives this device the same sensitivity limits as conventional PET devices.

The second approach is based on the use of a miniaturised probe, which is directly implantable into the cerebral tissue of the rodent. It includes a scintillating plastic optical fibre, which is connected via an optical guide to a low-noise photodetector. This probe enables the anaesthesia or restraint problems to be overcome. It has a high degree of sensitivity since it is placed directly in contact with the measurement region. Unlike external detector devices, which are based on the coincidence measurement of γ-ray annihilation of the positrons with the electrons, the probe directly measures the positrons (or β+ radiation) emitted, thereby improving the sensitivity of the detector and therefore the ability thereof to establish precise kinetics for the radioactive tracers. It can likewise be applied to the detection of β− or α radiation, thereby broadening the range of usable radioactive tracers. Finally, this type of device proves to be inexpensive and simple to use, in comparison with external detection devices.

Although this system proves to be very advantageous, it does not have any fewer limitations inherent in the technology used, since the use of a scintillating optical fibre as a radiation-sensitive sensor limits the measurement to a simple counting of the number of rays. Furthermore, the animal is not free to move during the measurement, because the photodetectors are too bulky to be placed on the animal, thereby requiring the photodetector to be placed off-centre and to be connected to the probe by an optical guide. Finally, the sensitivity of the sensor to light requires it to be used in darkness, thereby highly complicating the experimental conditions.

The objective of the invention, in particular, is to provide a simple, economical and effective solution to these various problems.

To that end, it proposes a device which is implantable into the brain of an animal, in particular, for detecting ionizing radiation emitted via spontaneous disintegration of a radioisotope, characterised in that it includes a needle-shaped implantable detector one end of which is intended to be implanted and bears at least one row of basic detectors oriented in the direction of implantation of the needle, the other end of the needle being secured to a printed circuit comprising a set of amplification, shaping, counting and wireless remote transmission circuits, which are connected to the basic detectors and which are intended to be worn by the animal, the latter being awake and free to move about.

The use of a semi-conductor material detector implanted inside the body of the animal enables direct conversion of the radiation energy derived from the disintegration into an electric current that is measurable in situ. The signal is then transmitted to an amplification circuit. A shaping circuit enables a portion of the parasitic signals resulting from electronic noise and parasitic photon noise to be eliminated. The signal is then transmitted remotely via a wireless connection for analysis and processing.

The needle can be formed from a substrate made of a high-resistivity semiconductor material bearing a plurality of electrodes forming the basic detectors.

According to another feature of the invention, each basic detector is connected to its own signal processing chain which forms part of an integrated circuit borne by the printed circuit and comprising means for amplifying, converting, filtering, thresholding and counting the signals from the basic detectors.

The connection of each basic detector to its own electronic chain enables time-dependent collection of the signals coming from the basic detectors, thereby making it possible to anticipate the post-production of biodistribution maps for the attachment of a radioactive tracer within the region analysed, and to follow the evolution of this distribution in order to carry out precise kinetic measurements owing to the improved sensitivity of the detector.

The entire device comprising the detector, which is implantable in the brain of the animal, as well as the various processing circuits, is intended to be worn by the animal, which remains entirely free in its movements, which limits the stress level of the animal considerably and facilitates the experiments.

The material used for the substrate and the detectors, for example, can be high-resistivity silicon. In this case, the basic detectors are reverse biased diodes. The use of a material having high resistivity enables the flow of leakage or parasitic currents within the diodes to be prevented.

The basic detectors are biased at an electric voltage enabling complete depletion of each basic detector.

The needle advantageously has a substantially rectangular cross-section and, on one of the faces thereof, holds the basic detectors. The length of the needle is of the order of 1 to 2 cm for a thickness of between 200 and 500 μm and a weight of less than 100 mg.

The exterior face of each basic detector and the face of the substrate opposite the basic detectors are each covered by a metal electrode.

The basic detectors have a width and a length of between 100 μm and 1 mm, e.g., a width of 200 μm and a length of 500 μm.

The basic detectors are connected to the amplification means by connecting tracks, which can be separated by grounded conducting lines, thereby enabling the crosstalk and capacitive coupling phenomena between the tracks to be limited.

According to another feature of the invention, a conducting ring surrounds all of the basic detectors and enables the electric field lines to be stabilised in the depletion region.

According to another feature of the invention, the printed circuit, detecting needle and integrated circuit assembly has a weight of less than 1 g. The surface area of the printed circuit is less than 1 cm².

The printed circuit is connected via a set of conductors, which can be subcutaneous, to an electric power supply, control and remote transmission module intended to be fastened to the back of the animal and having dimensions of the order of a centimetre.

The electrical power supply can be provided by means of battery cells, by radiofrequency, by photovoltaic cells or by photodiodes.

The remote transmission module can include a bidirectional radiofrequency system or an optical system, e.g., such as an infrared optical system.

According to another feature of the invention, the detector is covered by an impermeable, opaque, biocompatible and electrically insulating protective layer. Such a layer enables the detector to be protected from the moisture of the surrounding tissues and provides protection against the photons interfering with the detectors. The electrical insulation makes it possible to ensure optimal operation of each of the basic detectors and disturbance-free transmission of the signal to the processing circuit via the connecting tracks. The biocompatibility of the protective layer makes it possible to prevent potential inflammatory reactions, which can adversely affect the physiological parameters of the tissue studied and introduce a bias to the experiments.

This layer advantageously includes a first opaque and electrically insulating layer, and a second layer which covers the first and which is biocompatible and watertight. The first layer is a varnish-type coating and has a thickness of the order of a few micrometres, and the second layer is a plastic polymer of the polystyrene type and has a thickness of the order of 5 to 10 μm.

The device according to the invention is intended, in particular, for detecting β+, β− or α radiation for analysing the distribution and attachment of a radioactive tracer in the tissues with a temporal resolution of the order of one second. As a matter of fact, the detectors used in the device enable α or β-emitting radioactive tracers to be used, without being limited to the β+ emitting isotopes, which is beneficial to the development of new families of radioactive tracers. The device can be implanted into the brain of any type of animal and, in particular, into that of a small animal such as a rat or into a human brain.

The basic detectors arranged at one end of the needle can be of the CMOS or 3D type.

Other advantages and characteristics of the invention will become apparent upon reading the following description, which is given for non-limiting illustrative purposes and with reference to the appended drawings, in which:

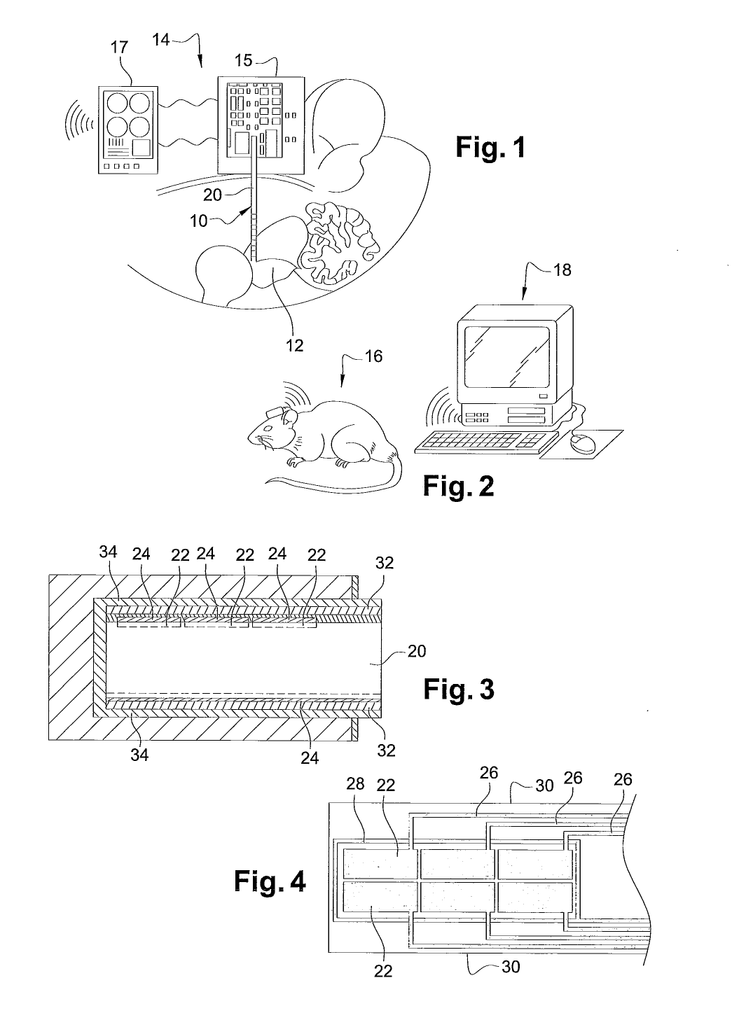

FIG. 1 is a schematic sectional view of the device according to the invention, comprising a detector implanted into the skull of an experimental animal;

FIG. 2 is a perspective schematic view of an experimental animal wearing the device of FIG. 1, which is connected to an analysis unit;

FIG. 3 is a schematic axial sectional view of the detector implanted into tissue;

FIG. 4 is a schematic top view of the detector of FIG. 3.

As shown in FIGS. 1 and 2, the device according to the invention includes means 10 for detecting β or α radiation, which are made of a semiconductor material implanted into the skull 12 of an experimental animal, and means 14 for processing the signal, some of which are mounted on a printed circuit 15 secured to the detection means 10 and connected to a module 17 for supplying power and for remote transmission to an analysis station 18, the module 17 being fastened to the animal a short distance away from the printed circuit 15.

The following description is made with reference to FIGS. 3 and 4. The detection means 10 include a needle 20 made of a semiconductor material the implanted end of which comprises a set of basic detectors 22, which are likewise made of a semiconductor material (only three basic detectors are visible in FIG. 3). The substrate forming the needle can be made of n-doped high-resistivity silicon, while the basic detectors 22 are p-doped so as to form a plurality of reverse-biased detection diodes. The bias voltage applied is such that it ensures complete depletion of each of the basic detectors 22, for the purpose of having a maximum detection volume for the radiation passing through the basic detectors 22.

The substrate 20 has a rectangular-shaped cross-section and the basic detectors 22 are held by one face of the substrate. The basic detectors 22 are aligned in the direction in which the detector 10 penetrates into the skull 12 of the animal 16.

The outside face of each basic detector 22 and the face of the substrate 20 opposite the basic detectors 22 are each covered by a metal electrode 24, made of aluminium and having a thickness of approximately 1 μm, for example.

FIG. 4 is a top view of the basic detectors 22 and a portion of the substrate 20 onto which they are fastened. Two parallel rows of three basic detectors 22 each are arranged at the implanted end of the needle. The basic detectors 22 have a rectangular shape and are aligned in the lengthwise direction thereof along the needle, so that the substrate 20 has a reduced cross-section in order to render the surgical operation of implanting the needle into the tissue as little traumatizing as possible for the animal 16.

The basic detectors 22 are connected via tracks 26 to processing means 14 borne by the printed circuit 15 and are surrounded as closely as possible by a conducting ring 28 which enables the electric field lines to be stabilised inside the active detection region of each of the basic detectors 22. The cut-out region 30 of the substrate is positioned at a sufficient distance from the ring 28 so as to minimise the leakage current phenomena resulting from the reduction in resistivity in the cut-out region 30, because of the modifications in the crystalline structure of the substrate at these locations. The distance between the cut-out region 30 and the ring typically corresponds to the thickness of the substrate, i.e., to the dimension of the cross-section of the substrate 20 in the direction perpendicular to the basic detectors 22. However, a compromise can be reached between compactness and leakage current, so as to have a distance between the ring 28 and the cut-out which is less than the thickness of the substrate 20.

The space between the conducting ring 28 and the cut-out region 30 is used for the passage of the various connecting tracks 26 to the basic detectors 22, thereby guaranteeing that the detector has a maximum degree of compactness.

Ground lines, not shown, can be made between each of the tracks 26 so as to limit the capacitive coupling or cross-talk phenomena between the tracks 26, which are made of a metallic material, and of aluminium, in a manner similar to the electrodes.

The invention enables operation of the device to be ensured under standard laboratory conditions, and in particular under normal lighting. To accomplish this, the substrate 20 is coated with an opaque layer 32, typically containing varnish, which ensures protection against the visible light reaching as far as the diodes. Such a layer likewise ensures electrical insulation of the basic detectors 22 and the tracks 26 thereof.

The detector is then coated with a second environmentally biocompatible layer 34 in which the detector is implanted. The desired protection is obtained by evaporating a polymer in an oven and by then re-polymerising on the detector coated with the first protective layer, in a chamber at ambient temperature. This method enables homogeneous deposition of the biocompatible layer 34 on the detector. The deposition of a polymer likewise ensures that the detector is protected against the moisture of the implantation medium, which can induce additional noise in the tracks 26 of the device. The thickness of this second layer must not be too significant, so as to not absorb the disintegration radiation. The polymer, for example, is polystyrene, and the thickness thereof is of the order of 5 to 10 μm.

The assembly formed by the printed circuit 15, the detection needle and the signal-processing means 14 borne by the circuit 15 has a weight of less than 1 g. The surface of the printed circuit 15 has a surface area of less than 1 cm².

The detector thus formed is pre-implanted into the study region by stereotaxis. The assembly is subsequently firmly attached to the skull of the animal, e.g., by a mechanical system such as a helmet or strap or else by cement. However, the user may choose to not attach the detector firmly in the case of specific short-term applications, and by leaving same fastened onto the stereotaxis system.

Each basic detector 22 is connected to its own signal-processing chain, which includes miniaturised amplification, conversion, filtering, thresholding and counting circuits which form part of an integrated circuit borne by the printed circuit 15, which is secured to the outside end of the detection needle and which is connected to the electrical power supply, control and wireless transmission module 17. The integrated circuit made using a sub-micronic technology is resistant to ionising radiation.

The signals transmitted by the tracks 26 to the printed circuit 15 are amplified by charge amplifiers and then converted into voltage. The thresholding circuits make it possible to allow only those signals corresponding to energy radiation higher than an operator-adjustable threshold to pass towards the counting circuits. This makes it possible to eliminate the electronic noise and to optimise the number of β particles counted in relation to the noise resulting from the parasitic photons.

Upon exiting the printed circuit, the digital signals are transmitted remotely by the module 17. The transmission can be carried out using a bidirectional radiofrequency system or an optical system such as an infrared optical system, for example.

The electrical power supply to the device can be provided by means of two 1.5-Volt battery cells enabling use of the detector over a long time period, e.g., of the order of about one hundred hours. Other types of power supply can be used, such as radiofrequency, photovoltaic cell or photodiode systems. A voltage-raising system can be used in order to obtain the bias voltage required by the detectors from the voltage delivered by the cells, which is of the order of a few tens of volts. This voltage-raising system can be provided by a charging pump. The electric power supply, control and remote transmission module 17 can be attached behind the implantation site for the detector, e.g., on the neck or back of the animal 16, via a small backpack or else by straps. This module has dimensions of the order of 1 cm², thereby guaranteeing complete freedom to the animal during the experiments.

The printed circuit 15 can be connected to the module 17 by a mini-cable, which can be external or subcutaneous in order to prevent it from being torn away by the animal during experiments.

The device operates in the following way: a radioactive tracer injected intravenously into the body of the animal attaches itself to a tissue of interest in proximity to the detector. The radiation emitted by spontaneous disintegration of the radioisotope passes through the detector and induces a deposit of charges via ionisation, which is proportional to the energy deposited by the radiation in the depleted region. The charging signal is transmitted to the processing circuits for amplification, conversion, filtering, thresholding and counting, and is then transmitted to an analysis and post-processing unit 18 situated several metres from the animal 16, for example.

Numerous alternatives to the device described can be anticipated. For example, it is possible to use a larger number of basic detectors 22 than that shown in the drawings. The needle, for example, can thus comprise two rows of 10 detectors each.

The substrate 20 typically has a thickness of the order of 200 to 500 μm, a width of the order of 1 mm, and a length of the order of 1 to 2 cm for a weight of less than 100 mg. The basic detectors have a width and a length of between 100 μm and 1 mm.

In one particular embodiment of the invention, the thickness of the substrate is of the order of 500 μm, the width and the length of the basic detectors being 200 μm and 500 μm, respectively.

The distance between detectors is of the order of 20 μm and the distance separating two connecting tracks is of the order of 10 μm.

The bias voltage of the basic detectors 22 is of the order of a few tens of volts.

The semiconductor material can be made of high-resistivity silicon.

The device according to the invention enables the detection of β+ radiation, and α or β− radiation, depending on the radioisotope used. It enables accurate measurement of the temporal evolution of the radiation activity in a tissular region of interest, for a subject who is awake and completely free to move about, owing to the extreme compactness of the detector coupled to completely self-contained miniature electronics. In addition, the direct detection of the β radiation, instead of the γ radiation resulting from the annihilation process, enables sensitivity to be improved and a temporal resolution of the order of a second to be obtained, thereby improving the accuracy of the kinetic measurements. Finally, the possibility of detecting not only the β+ radiation, but likewise the β− and α radiation, enables the field of application of the device to be extended to new α or β− emitting radioactive tracers.

The device according to the invention can be used in combination with a second identical device, one of the devices being implanted in a study region, the other being implanted in a control region. The analysis of the kinetic evolution of the difference in the signal coming from the control region with the signal coming from the study region enables information to be obtained about the level of biological activity specific to the study region.

The device according to the invention is not limited to the functional exploration of the tissues of the neurocranium. As a matter of fact, it is entirely possible to implant a detector in the tissues of other organs of the animal where it is desired to evaluate the attachment of a radioactive tracer. In the same way, the device according to the invention can be used on animals of a smaller size than rodents, or on man.

The device according to the invention can likewise be used in combination with basic detectors of the CMOS (Complementary Metal Oxide Semi-Conductor) type, the dimensions of which can be reduced to values of the order of 10 μm. In this case, the bias voltage applied to the basic detectors can be lower than with diodes, and the device does not require any conducting ring 28.

The device according to the invention can likewise be used in combination with semiconductor detectors of the “3D” type, where the electrodes which establish the electric field of depletion are plated-through holes made in the substrate. In this case, the device does not require any conducting ring 28.

1. Device which is implantable into the brain of an animal, in particular, for detecting ionizing radiation emitted via spontaneous disintegration of a radioisotope, wherein it includes a needle-shaped implantable detector one end of which is intended to be implanted and bears at least one row of basic detectors oriented in the direction of implantation of the needle, the other end of the needle being secured to a printed circuit comprising a set of amplification, shaping, counting and wireless remote transmission circuits, which are connected to the basic detectors and which are intended to be worn by the animal, the latter being awake and free to move about.

2. Device of claim 1, wherein the needle is formed from a substrate made of a high-resistivity semiconductor material bearing a plurality of electrodes forming the basic detectors.

3. Device as claimed in claim 1, wherein each basic detector is connected to its own signal processing chain which forms part of an integrated circuit borne by the printed circuit and comprising means for amplifying, converting, filtering, thresholding and counting the signals from the basic detectors.

4. Device of claim 3, wherein the assembly of the needle, the printed circuit and the signal-processing integrated circuit has a weight of less than 1 g, the printed circuit having a surface area of less than 1 cm2.

5. Device as claimed in claim 3, wherein the basic detectors are biased at an electric voltage enabling complete depletion of each basic detector.

6. Device as claimed in claim 3, wherein the needle has a substantially rectangular cross-section and, on one of the faces thereof, holds the basic detectors.

7. Device of claim 6, wherein the exterior face of each basic detector and the face of the substrate opposite the basic detectors are each covered by a metal electrode.

8. Device as claimed in claim 6, wherein the basic detectors have a width and a length of between 100 μm and 1 mm, e.g., a width of 200 μm and a length of 500 μm.

9. Device as claimed in claim 3, wherein the needle has a length of the order of 1 to 2 cm, a thickness of between 200 and 500 μm and a weight of less than 100 mg.

10. Device as claimed in claim 3, wherein the connecting tracks connect the basic detectors to the amplification means and are separated by grounded conducting lines.

11. Device as claimed in claim 3, wherein a conducting ring surrounds all of the basic detectors.

12. Device as claimed in claim 3, wherein the printed circuit secured to the needle is connected via a set of conductors to an electric power supply, control and remote wireless transmission module intended to be fastened onto the back of the animal or another portion of the body thereof and having dimensions of the order of a centimetre.

13. Device as claimed in claim 3, wherein the printed circuit secured to the needle is intended to be connected to the rower supply, control and transmission module via a subcutaneous wire connection.

14. Device as claimed in claim 12, wherein the power supply is provided by means of battery cells, by radiofrequency, by photovoltaic cells or by photodiodes.

15. Device as claimed in claim 12, wherein the remote transmission module includes a bidirectional radiofrequency system or an optical system, e.g., such as an infrared optical system.

16. Device as claimed in claim 2, wherein the substrate and the basic detectors are made of high-resistivity silicon.

17. Device as claimed in claim 1, wherein the detector covered by an impermeable, opaque, biocompatible and electrically insulating protective layer.

18. Device of claim 17, wherein the protective layer includes a first opaque and electrically insulating layer, and a second layer which covers the first and which is biocompatible and watertight.

19. Device of claim 18, wherein the first layer is a varnish-type coating and has a thickness of the order of a few micrometres, and the second layer is a plastic polymer of the polystyrene type and has a thickness of the order of 5 to 10 μm.

20. Device as claimed in claim 1, wherein it is intended for detecting β+, β− or α radiation for analysing the distribution and attachment of a radioactive tracer in the tissues with a temporal resolution of the order of one second.

21. Device as claimed in claim 1, wherein it is intended to be implanted into the brain of a small animal such as a rat, for example, or into the brain of a large animal, or into a human brain.

22. Device as claimed in claim 1, wherein the basic detectors are of the CMOS or 3D type.
